(19) United States
(12) Patent Application Publication
(10) Pub. No.: US 2007/0203386 A1
(43) Pub. Date: Aug. 30, 2007

(54) Process for the preparation of and composition of a feedstock usable for the preparation of lower olefins

(76) Inventor: Luis Pablo Dancuart Kohler, Vaalpark (ZA)

Correspondence Address: KNOBBE MARTENS OLSON & BEAR LLP, 2040 MAIN STREET, FOURTEENTH FLOOR, IRVINE, CA, 92614, US

(21) Appl. No.: 11/707,366

(22) Filed: Feb. 15, 2007

Related U.S. Application Data
(62) Division of application No. 10/358,129, filed on Jan. 31, 2003, now abandoned.

(51) Int. Cl. C07C 4/04

(52) U.S. Cl. 585/648

(57) ABSTRACT

The invention provides a process for the preparation of lower olefins by a thermal cracking process, a process for the preparation of a synthetic hydrocarbon feedstock for such a process, and a composition of a high performance feedstock usable in said process.

### CROSS-REFERENCE TO RELATED APPLICATIONS

This application is a division of Ser. No. 10/358,129, filed Jan. 31, 2003, the disclosure of which is hereby expressly incorporated by reference in its entirety and is hereby expressly made a portion of this application.

### FIELD OF THE INVENTION

The invention relates to a process for the preparation of lower olefins and a composition of a high performance feedstock usable in a process for the preparation of lower olefins from a hydrocarbon containing feed including at least a fraction boiling above the boiling point range of the lower olefins. In particular, this feedstock can be used advantageously when the lower olefins process objective is propylene.

### BACKGROUND TO THE INVENTION

The thermal cracking process for production of lower olefins, sometimes also known as steam cracking or pyrolysis, is the most important commercial route for the production of ethylene, propylene and other lower olefins from a hydrocarbon containing feed including at least a fraction above the boiling point range of the lower olefins.

The chemical reactions that occur during thermal cracking are increasingly more complex when processing heavy feedstock at high conversion. Naphthas are regarded as the lightest of the heavy feedstocks suitable for thermal cracking.

The thermal cracking of naphtha feedstocks proceeds in two stages. The primary reactions that take place in the first stage include the thermal decomposition of the reactants by free-radical chain mechanism into hydrogen, methane, ethylene, propylene, butylenes and higher olefins. The second stage involves three types of reactions:
a. further thermal cracking of the olefins derived from the primary reactions;
b. production of paraffins, diolefins and alkynes from the same olefins via hydrogenation and dehydrogenation; and
c. condensation reactions to aromatics and cyclodiolefins molecules which are stable and, ultimately, lead to coke.

Thus, the applicant is aware of EP 0 584 879 B1 in which it is disclosed that a hydroprocessed synthetic oil fraction may be cracked using a thermal cracking process to increase the selectivity of the cracking process to lower olefins when compared to the thermal cracking of unhydroprocessed synthetic oil fractions or crude oil derived hydrocarbon feeds.

The ideal characteristics of a synthetic naphtha usable for the production of lower olefins have been defined in an earlier paper by Frohning and Cornills (Hydrocarbon Processing, Nov. 1974, p.143-146). They indicated that the high content of unsaturated compounds, including olefinic species reduces the versatility of the synthetic naphthas as a cracking feedstock unless part of the unsaturation is removed by hydrogenation. They indicated that the ideal target would be a residual content of 10-15% olefins. Surprisingly it has been found that a synthetic naphtha with an olefin contents above this range performed as a cracking feedstock better than a fully hydrogenated synthetic naphtha.

In this specification reference is made to Fischer-Tropsch (FT) reactions, FT reactors, FT products, and the like. The FT process is a well known process in which carbon monoxide and hydrogen are reacted over an iron, cobalt, nickel or ruthenium containing catalyst to produce a mixture of straight and branched chain hydrocarbons ranging from methane to waxes and smaller amounts of oxygenates.

The FT process is used industrially to convert synthesis gas, which might derived from coal, natural gas, biomass or heavy oil streams, into hydrocarbons ranging from methane to species with molecular masses above 1400.

While the main products are linear paraffinic materials, other species such as branched paraffins, olefins and oxygenated components may form part of the product slate. The exact product slate depends on reactor configuration, operating conditions and the catalyst that is employed, as is evident from articles such as Catal.Rev.-Sci. Eng., 23(1&2), 265-278 (1981).

Preferred reactors for the production of heavier hydrocarbons are slurry bed or tubular fixed bed reactors, while operating conditions are preferably in the range of 160-280° C., in some cases in the 210-260° C. range, and 18-50 bar, in some cases preferably between 20-30 bar.

The catalyst may comprise active metals such as iron, cobalt, nickel or ruthenium. While each catalyst will give its own unique product slate, in all cases the product slate contains some waxy, highly paraffinic material which needs to be further upgraded into usable products. The FT products can be hydroconverted into a range of final products, such as middle distillates, naphtha, solvents, lube oil bases, etc. Such hydroconversion, which usually consists of a range of processes such as hydrocracking, hydrotreatment and distillation, can be termed a FT products work-up process.

In an ongoing search for alternative feedstock and/or better process yields for the preparation of lower olefins the applicant now proposes the following invention.

### SUMMARY OF THE INVENTION

The invention provides a process for the preparation of lower olefins from a synthetic hydrocarbon feed including at least a fraction having a boiling point above the boiling point range of the lower olefins, wherein the hydrocarbon feed includes an unhydrogenated fraction, which process includes the thermal processing of the hydrocarbon feed.

The thermal processing may be thermal cracking of the synthetic hydrocarbon feed under thermal cracking conditions selected to suit the synthetic hydrocarbon feed composition.

Typically the thermal cracking conditions are a temperature of from 400° C. to 1200° C., usually from 700° C. to 950° C. and at a pressure of from 0.1 to 20 bar absolute pressure, typically from 1 to 5 bar.

The residence time in a thermal cracking unit in which the thermal cracking is taking place may be from 50 ms to 1000 ms, or even longer. Typically the residence time may be below 300 ms. The residence time will however depend on the configuration of the thermal cracking unit used.

The thermal cracking may be carried out in the presence of an inert media. This inert media might be steam or nitrogen.

The synthetic hydrocarbon feed may be the product of a FT reaction.

The synthetic hydrocarbon feed may be the processed product of the FT reaction.

The unhydrogenated fraction of the synthetic hydrocarbon feed may be an unhydrogenated fraction of the process products of the FT reaction.

The synthetic hydrocarbon feed may be prepared by combining at least
an unhydrogenated fraction of the process products of the FT reaction, also referred to as an unhydrogenated FT fraction; and
a hydroconverted fraction of the process products of the FT reaction, also referred to as a hydroprocessed or hydrocracked FT fraction.

The unhydrogenated FT fraction may include a fraction of the condensate product of the FT reaction The FT condensate is typically obtained as the liquid hydrocarbon stream from the FT products excluding the FT wax from a FT reactor in which the FT reaction has taken place.

The hydroprocessed FT fraction may be a hydrocracked FT wax fraction of the FT reaction products. The FT wax fraction is typically obtained as the heavy hydrocarbons stream.

The invention extends to a hydrocarbon feed to a cracking process for the preparation of lower olefins from a synthetic hydrocarbon feed including at least a fraction having a boiling point above the boiling point range of the lower olefins, said hydrocarbon feed including at least a fraction which is unhydrogenated.

The synthetic hydrocarbon feed may be the product of a FT reaction.

The synthetic hydrocarbon feed may be the processed product of the FT reaction.

It has been found that the synthetic hydrocarbon feed is optimised when the target product of the cracking process is propylene. Moreover, it has also been found that this synthetic hydrocarbon feed can yield significantly lower yields of the undesirable liquid product (C5+ fraction) that is co-produced during cracking to lower olefins.

According to a further aspect of the invention there is provided a thermal process for the preparation of lower olefins from a synthetic hydrocarbon feed including at least a fraction having a boiling point above the boiling point range of the lower olefins, wherein the hydrocarbon feed comprises at least 15% olefins. This synthetic hydrocarbon feed, also referred to as olefinic naphtha, also has a low aromatics content, typically below 1% mass, preferably below 0.5% mass. This is believed to be a contributing factor to the superior thermal cracking performance.

The invention extends to a hydrocarbon feed to a cracking process for the preparation of lower olefins from a synthetic hydrocarbon feed including at least a fraction having a boiling point above the boiling point range of the lower olefins, said hydrocarbon feed comprising at least 15% olefins and at most 1.0% aromatics.

The hydrocarbon feed may comprise at least about 20% olefins .

According to an aspect of the invention, there is provided a process for the preparation of a synthetic hydrocarbon feed to a process for producing lower olefins, said feed including at least a fraction having a boiling point above the boiling point range of the lower olefins, said process including the steps of:
a) fractionating a straight run unhydrogenated condensate fraction of a FT synthesis product of H₂ and CO to obtain a synthetic olefinic naphtha;
b) hydroconverting by a process including hydrocracking at least a wax fraction of the FT synthesis product of H₂ and CO, or a derivative thereof;
c) fractionating the hydroconverted wax product from step b) to obtain a hydroconverted naphtha fraction separated from the other products from the hydroconversion process; and
d) blending said olefinic naphtha from step a) with the hydroconverted naphtha from step c) to obtain a a synthetic hydrocarbon feed in a desired ratio having a boiling point above the boiling point range of the lower olefins.

The wax fraction of step b) may have a true boiling point (TBP) in the range of about 70° C. to 700° C., typically in the range 80° C. to 650° C.

The FT condensate fraction of step a) may have a true boiling point (TBP) in the range −70° C. to 350° C., typically −10° C. to 340° C., usually −70° C. to 350° C.

Typically, the hydroconverted product of step c) is blended with the synthetic hydrocarbon fraction of step a) in a volumetric ratio of from 1:4 to 4:1 to form the synthetic hydrocarbon feed of step d). Typically this volumetric ratio is 1:2 to 2:1, even more typically between 3:2 and 2:3.

According to a further aspect of the invention, there is provided a process for the preparation of a synthetic hydrocarbon feed for a process for producing lower olefins, said feed including at least a fraction having a boiling point above the boiling point range of the lower olefins, said process including the steps of:
a) hydroconverting by a process including hydrocracking at least a wax fraction of the FT synthesis product of H₂ and CO, or a derivative thereof;
b) blending said hydroconverted product from step a) with a straight run unhydrogenated condensate fraction in a desired ratio to obtain a blend that includes hydrocarbons boiling over a broad temperature range; and
c) fractionating the hydrocarbons blend from step b) to obtain a synthetic hydrocarbon feed for the thermal cracking process having a boiling point above the boiling point range of the lower olefins.

The hydroconverted product of step a) may be blended with the condensate hydrocarbon fraction in step b) in a volumetric ratio of from 1:10 to 10:1 before being fractionated in a single unit to form the synthetic hydrocarbon feed of step c).

The wax fraction of step a) may have a true boiling point (TBP) in the range of about 70° C. to 700° C., typically in the range 80° C. to 650° C.

The FT condensate fraction of step b) may have a true boiling point (TBP) in the range −70° C. to 350° C., typically −10° C. to 340° C., usually −70° C. to 350° C.

The synthetic hydrocarbon fraction usable as feedstock for thermal cracking may be a C5 to 160° C. boiling range, defined based on the ASTM D86 Distillation standard, synthetic naphtha.

According to a further aspect of the invention, it is believed that a semi-synthetic feedstock may be produced which is usable for the production of lower olefins via thermal cracking, said feedstock comprising an olefinic synthetic feedstock obtained from a FT synthesis product of H₂ and CO and a highly paraffinic fraction selected from a petroleum liquid fraction and a natural gas liquid fraction, blended to have at least a 15% mass olefins content and an aromatics content below 1%.

The highly paraffinic naphtha may be a product obtained from conventional petroleum refining scheme or from the fractionation of the liquid hydrocarbons contained in natural gas. The blending ratio required is selected for each specific highly paraffinic naphtha in order to obtain a semi-synthetic naphtha with a similar olefins content as that of the above described fully synthetic naphtha, i.e. in the range of more than 15% or more than 20% by mass together with an aromatics content below 1% mass.

### BRIEF DESCRIPTION OF THE DRAWINGS

FIG. 1 depicts a basic process for the preparation of synthetic hydrocarbon feed.

FIG. 2 depicts an alternative process for the preparation of a synthetic hydrocarbon feed wherein the lighter Fischer-Tropsch condensate is recovered and is sent to a common fractionator unit with a hydroprocessed product.

### DETAILED DESCRIPTION OF THE PREFERRED EMBODIMENT

Table 1 gives a typical composition of the two FT liquid fractions obtainable from a FT reactor.

TABLE 1
Typical FT product after separation into two fractions
(vol % distilled)

| Distillation | FT Condensate | FT Wax |
|---|---|---|
| Range | (<270° C. fraction) | (>270° C. fraction) |
| C₅-160° C. | 44 | 3 |
| 160-270° C. | 43 | 4 |
| 270-370° C. | 13 | 25 |
| 370-500° C. | — | 40 |
| >500° C. | — | 28 |

Catalysts for the FT wax hydroprocessing are typically of the bifunctional type; i.e. they contain sites active for cracking and for hydrogenation. Catalytic metals active for hydrogenation include group VIII noble metals, such as platinum or palladium, or a sulphided Group VIII base metals, e.g. nickel, cobalt, which may or may not include a sulphided Group VI metal, e.g. molybdenum. The support for the metals can be any refractory oxide, such as silica, alumina, titania, zirconia, vanadia and other Group III, IV, VA and VI oxides, alone or in combination with other refractory oxides. Alternatively, the support can partly or totally consist of zeolite. However, for this invention the preferred support is amorphous silica-alumina.

Process conditions for hydrocracking can be varied over a wide range and are usually laboriously chosen after extensive experimentation to optimise the yield of naphtha. In this regard, it is important to note that, as in many chemical reactions, there is a trade-off between conversion and selectivity. A very high conversion will result in a high yield of gases and low yield of naphtha fuels. It is therefore important to painstakingly tune the process conditions in order to optimise the conversion of >160° C. hydrocarbons. Table 2 gives a list of one such set of conditions.

TABLE 2
Process conditions for hydrocracking of the FT Wax

| | BROAD | PREFERRED |
|---|---|---|
| CONDITION | RANGE | RANGE |
| Temperature, ° C. | 150-450 | 340-400 |
| Pressure, bar-g | 10-200 | 30-80 |
| Hydrogen Flow Rate, m³ₙ/m³ feed | 100-2000 | 800-1600 |
| Conversion of>370° C. material, mass % | 30-80 | 50-70 |

Nevertheless, it is possible to convert all the >370° C. material in the feedstock by recycling the part that is not converted during the hydrocracking process.

### DESCRIPTION OF EXAMPLES OF THE INVENTION

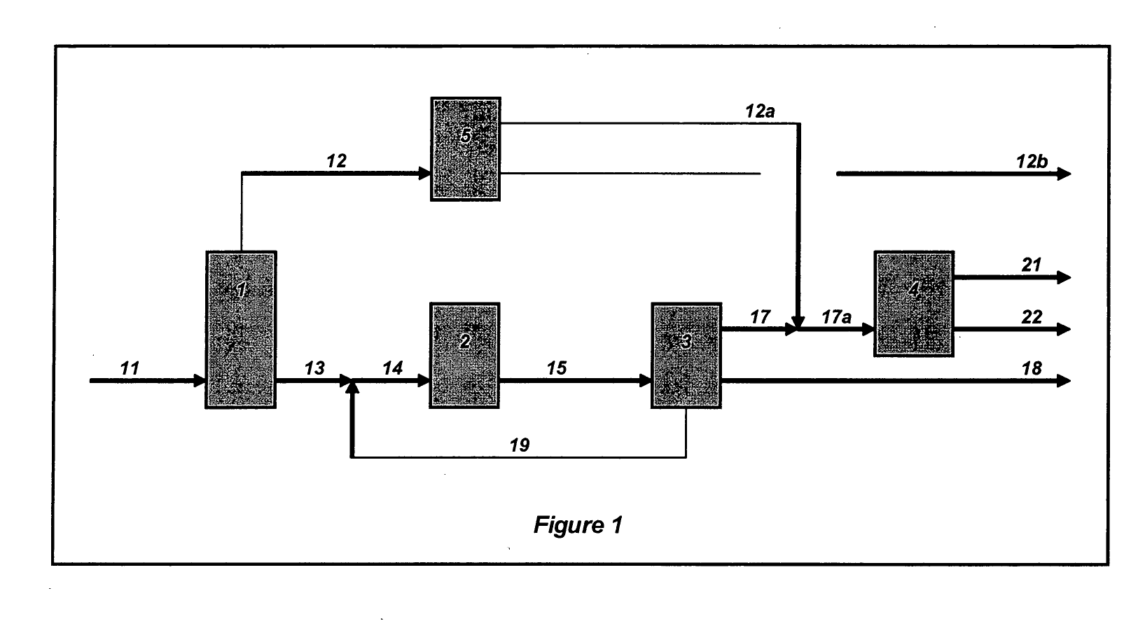

A basic process for the preparation of synthetic hydrocarbon feed is outlined in FIG. 1. A synthesis gas (syngas) stream 11, a mixture of hydrogen and carbon monoxide, enters the FT reactor 1 where the synthesis gas is converted to hydrocarbons by the FT reaction. A lighter unhydrogenated FT fraction i.e. the FT condensate, is recovered in line 12, and is sent to be fractionated to fractionator unit 5 where an olefinic naphtha fraction is recovered in line 12a together with an olefinic diesel product via line 12b.

A waxy FT fraction is recovered in line 13 and sent to the hydroconversion unit 2. The hydroprocessed product is sent to fractionator unit 3 where at least three products are recovered: hydroprocessed naphtha 17, hydroprocessed diesel 18, and unconverted waxy species that might be recycled to unit 2 via line 19.

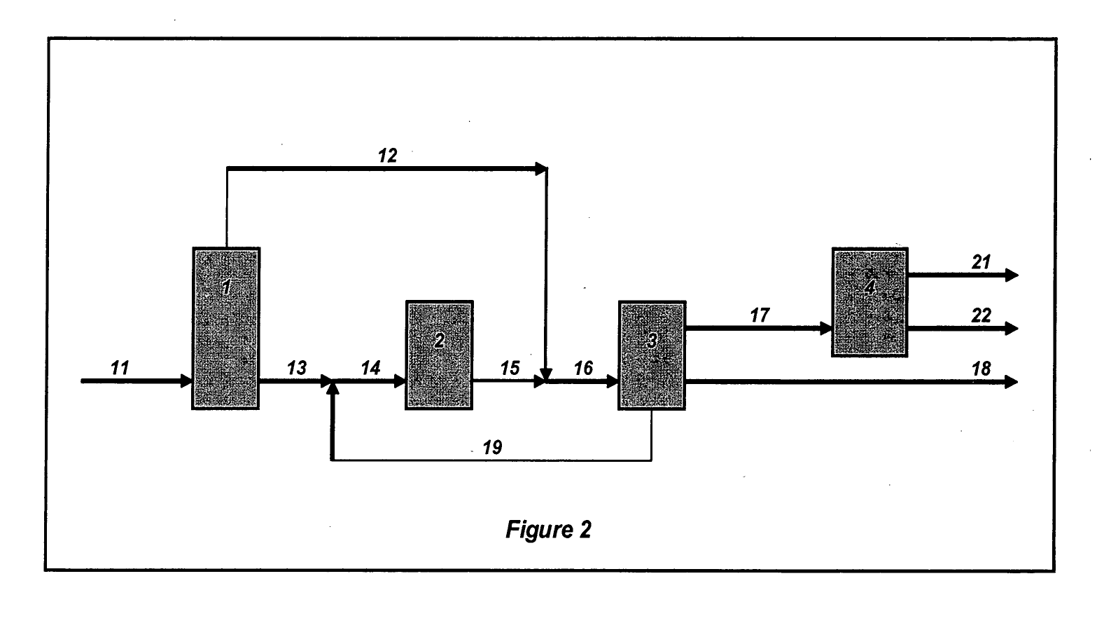

An alternative process is presented in FIG. 2 where the lighter FT fraction i.e. the FT condensate, is recovered in line 12, and is sent to be fractionated to common fractionator unit 3 with the hydroprocessed product from the hydroprocessing unit 2.

A small amount of C₁-C₄ gases are also separated in fractionator 3 and, if included a second fractionator 5. While these are not shown in this description, this simplification should be clear to any person skilled in the art.

The synthetic hydrocarbon feed of this invention may be produced either by blending streams 12a and 17 (as shown in FIG. 1) when two fractionators are included in the process scheme or as a single stream 17 (as shown in FIG. 2) when only one fractionator is used. The synthetic hydrocarbon feed is a naphtha product which comprises typically a C₅-160° C. fraction and are useful as a petrochemical naphthas.

The blending ratio for naphtha streams 12a and 17, obtainable when including two fractionators can range from 1:2 to 2:1, typically the ratio is between 2:3 and 3:2.

The blending ratio for naphtha 12 and hydro processed wax 15 can range from 1:10 to 10:1.

Either of these naphtha products can then be thermally cracked in thermal cracking unit 4 where the lower olefins are to be produced. For simplicity reasons only two product streams are shown exiting unit 4. Stream 21 contains the lower olefins and stream 22 contains all other thermal cracking products. This simplification should be clear to any person skilled in the art.

A somewhat heavier cut, synthetic diesel is also obtainable by blending streams 12b (olefinic distillate) and 18 (hydroconverted distillate) when two fractionators are included (FIG. 1) when two distillate fractions are recovered. Alternatively, an equivalent product can be recovered as stream 18 as shown in FIG. 2. These two products can be used on their own or blended as a single product. All of these distillate cuts are typically recovered as 165-370° C. fractions useful as diesel fuel.

In either case the heavy unconverted material 19 from fractionator 3 is typically recycled to extinction to hydroconversion unit 2. Alternatively, the residue may be used for production of high viscosity index synthetic lube oil bases.

Experimental Data

The synthetic hydrocarbon feed including an unhydrogenated FT fraction was prepared using the process as described above as was a fully hydroprocessed synthetic hydrocarbon feed for comparative testing. The characteristics of these two feeds are set out in Table 3. Naphtha 1 is a fully hydroprocessed FT naphtha with a 0.5% residual olefins content. Naphtha 2 is an olefinic FT naphtha prepared using a process scheme such as that described in FIG. 2. Its olefins content was 21.9%.

TABLE 3
Characteristics of the Synthetic Hydrocarbons Feeds

| | | Naphtha 1 | Naphtha 2 |
|---|---|---|---|
| Specific Gravity | | 0.691 | 0.703 |
| ASTM D86 Distillation | T0, ° C. | 51 | 65 |
| | T5, ° C. | 54 | 70 |
| | T10, ° C. | 57 | 75 |
| | T30, ° C. | 75 | 91 |
| | T50, ° C. | 93 | 99 |
| | T70, ° C. | 107 | 113 |
| | T90, ° C. | 125 | 130 |
| | T95, ° C. | 130 | 132 |
| | T100, ° C. | 134 | 135 |
| Composition | | | |
| Alkanes | % | 91.9% | 71.9% |
| Olefins | % | 0.5% | 21.9% |
| Naphthenes | % | 7.1% | 3.5% |
| Aromatics | % | 0.5% | 0.3% |
| Oxygenates | % | ND | 2.4% |
| Total | % | 100.0% | 100.0% |
| Sulfur | ppm | <1 | <1 |
| Total Oxygen | ppm | NA | 3405 |

The Cracking severity was measured by calculating the Propylene/Ethylene (P/E) mass ratio. Note that P/E ratio has an inverse relation to the cracking severity: high P/E ratios correspond to low severities (i.e. lower cracking temperatures) and lower methane co-produced.

These two hydrocarbon feeds were then subjected to thermal cracking as set out in table 4 below. In the comparison of performance, the cracking severity as measured by the P/E mass ratio was within ±0.01. This difference value can be regarded as acceptable to considered the result sets as equivalent cases. It is important to highlight that the economics of commercial scale cracking to lower olefins can be significantly affected by improved product yields, even as low as 0.5-1.0% percentage points.

Conventionally, the lower olefins of commercial interest are ethylene, propylene and butadiene. The thermal cracking process also results in the production of unsaturated liquid hydrocarbons with five or more carbon atoms (C5). This product is of low commercial interest to thermal cracker operators.

The results presented in examples 1 and 2 clearly indicate that the olefinic Naphtha 2 resulted in a better performance compared with fully hydroprocessed Naphtha 1 when thermally cracked. This was particularly true for the following performance criteria: (1) Higher ethylene and propylene yields, and consequently higher lower olefin yields, and (2) Lower yield of the unattractive thermal cracking liquids (C5+ fraction).

Therefore, the overall performance of the synthetic partially hydroconverted thermal cracking feed was better than that of its fully hydroprocessed counterpart.

### Example 1

### Low Cracking Severity—P/E ratio 0.59-0.60

The experimental results shown in Table 4 indicate that the Naphtha 2 resulted in 2.2% mass more ethylene that the fully saturated Naphtha 1—33.0% and 32.3% mass respectively. Similarly, the propylene yield was 3.1% higher, 19.7% and 19.1% respectively. On the same basis, the combined yields of the commercially attractive lower olefins, i.e. ethylene, 20 propylene and butadiene, was 3.6% higher—57.2% and 55.2% mass respectively. Finally, the yield of undesirable combined liquid products (C5+ material) was 11.2% lower when processing Naphtha 2, 15.0% and 16.9% respectively.

### Example 2

### High Cracking Severity—P/E ratio 0.50-0.51

The experimental results shown in Table 4 indicate that the Naphtha 2 resulted in 1.7% mass more ethylene that the fully saturated Naphtha 1—36.3% and 35.7% mass respectively. Similarly, the propylene yield was 3.9% higher, 18.5% and 17.8% respectively. On the same basis, the combined yields of the commercially attractive lower olefins, i.e. ethylene, propylene and butadiene, was 2.9% higher—59.4% and 57.7% mass respectively. Finally, the yield of undesirable combined liquid products (C5+ material) was 15,3% lower when processing Naphtha 2, 12.2% and 14.4% respectively.

TABLE 4
Steam Cracking Performance of Synthetic Hydrocarbon Feeds

| | | Naphtha 1 | | Naphtha 2 | |
|---|---|---|---|---|---|
| Propylene-to-Ethylene ratio | wt | 0.59 | 0.50 | 0.60 | 0.51 |
| Average Coil Temp., ° C. | ° C. | 868 | 892 | 877 | 896 |
| Product Yields (mass %) | | | | | |
| Ethylene | wt% | 32.3 | 35.7 | 33.0 | 36.3 |
| Propylene | wt% | 19.1 | 17.8 | 19.7 | 18.5 |
| 1,3-Butadiene | wt% | 3.8 | 4.2 | 4.5 | 4.7 |
| Total Lower Olefins | wt% | 55.2 | 57.7 | 57.2 | 59.4 |
| Other Olefins | wt% | 8.4 | 6.2 | 7.6 | 5.8 |
| Hydrogen | wt% | 0.7 | 0.8 | 0.7 | 0.8 |
| Methane | wt% | 13.0 | 14.9 | 13.4 | 15.3 |
| Other Paraffins | wt% | 5.3 | 4.8 | 5.3 | 5.2 |
| Alkynes | wt% | 0.7 | 1.1 | 0.8 | 1.1 |
| CO/CO2 | wt% | 0.0 | 0.1 | 0.0 | 0.1 |
| C5 + Liquids | wt% | 16.9 | 14.4 | 15.0 | 12.2 |
| Total Products | wt% | 100.0 | 100.0 | 100.0 | 100.0 |

### Example 3

### Preparation of a Semi-synthetic Naphtha

A semi-synthetic naphtha whose olefin content is similar to that of the olefinic Naphtha 2 can be prepared by blending the FT straight run Naphtha 3 fractionated from the FT condensate with a highly paraffinic conventional petrochemical naphtha. The composition of two of 10 these products is presented in Table 5.

TABLE 5
Components of a Semi-synthetic Olefinic Naphtha

| | Paraffinic | Blend (% mass of Naphtha 3) | | | | | Naphtha |
|---|---|---|---|---|---|---|---|
| | Naphtha | 20% | 40% | 50% | 60% | 80% | 3 |
| Density, | 0.660 | 0.669 | 0.679 | 0.684 | 0.689 | 0.699 | 0.710 |
| kg/L (20° C.) | | | | | | | |
| Total sulphur, | <2 | <2 | <2 | <1 | <1 | <1 | <1 |
| ppm | | | | | | | |

Composition, % wt

| | | | | | | | |
|---|---|---|---|---|---|---|---|
| Paraffins | 93.0 | 85.3 | 77.6 | 73.7 | 69.8 | 62.1 | 54.4 |
| Naphthenics | 5.5 | 4.4 | 3.3 | 2.7 | 2.2 | 1.1 | 0.0 |
| Aromatics | 1.5 | 1.2 | 0.9 | 0.8 | 0.6 | 0.3 | 0.0 |
| Olefins | 0.0 | 7.0 | 14.0 | 17.5 | 21.0 | 28.0 | 35.0 |
| Oxygenates | 0.0 | 2.1 | 4.3 | 5.3 | 6.4 | 8.5 | 10.7 |
| Total | 100.0 | 100.0 | 100.0 | 100.0 | 100.0 | 100.1 | 100.1 |

It is evident that, in this case, the target of ca 20% olefins is obtainable for blends of ca 55% Naphtha 3 with the balance being a highly paraffinic petrochemical naphtha.

What is claimed is:

1. A thermal cracking process for the preparation of lower olefins from a synthetic hydrocarbon feed, the process comprising the step of thermally cracking a synthetic hydrocarbon feed in the presence of an inert medium to obtain lower olefins, wherein the synthetic hydrocarbon feed comprises at least one fraction having a boiling point above a boiling point of the lower olefins, wherein the synthetic hydrocarbon feed has an olefins content of at least 15 mass %, and wherein the synthetic hydrocarbon feed has an aromatics content of below 1 mass %.

2. The process of claim 1, wherein the synthetic hydrocarbon feed is a product of a Fischer-Tropsch reaction.

3. The process of claim 1, wherein the synthetic hydrocarbon feed is prepared by combining at least one unhydrogenated fraction of a Fischer-Tropsch reaction product and a hydroconverted fraction of a Fischer-Tropsch reaction product.

4. The process of claim 3, wherein the unhydrogenated fraction is a condensate fraction of a Fischer-Tropsch reaction product.

5. The process of claim 3, wherein the hydroconverted fraction is a hydro cracked wax fraction of a Fischer-Tropsch reaction product.

6. The process of claim 1, wherein the synthetic hydrocarbon feed has an olefins content of at least about 20 mass %.

7. The process of claim 1, wherein the synthetic hydrocarbon feed has an aromatics content of below 0.5 mass %.

8. The process of claim 1, wherein the inert medium is nitrogen.

9. The process of claim 1, wherein the inert medium is steam.

10. The process of claim 1, wherein the lower olefins are selected from the group consisting of ethylene, propylene, butadiene, and mixtures thereof.

11. The process of claim 1, wherein the step of thermally cracking is conducted at a temperature of from 400° C. to 1200° C. and at a pressure of from 0.1 bar absolute pressure to 20 bar absolute pressure.

12. The process of claim 1, wherein the step of thermally cracking is conducted in a thermal cracking unit at a residence time of from 50 ms to 1000 ms.

13. A thermal cracking process for the preparation of lower olefins from a semi-synthetic hydrocarbon feed, the process comprising the step of thermally cracking a semi-synthetic hydrocarbon feed in the presence of an inert medium to obtain lower olefins, wherein the semi-synthetic hydrocarbon feed comprises an olefinic synthetic feedstock obtained from a Fischer-Tropsch synthesis product of H₂ and CO; wherein the semi-synthetic hydrocarbon feed further comprises a highly paraffinic fraction selected from the group consisting of a petroleum liquid fraction, a natural gas liquid fraction, and mixtures thereof; and wherein the olefinic synthetic feed stock and the highly paraffinic fraction are blended such that the semi-synthetic hydrocarbon feed has at least a 15 mass % olefins content and an aromatics content below 1 mass %.

14. The process of claim 13, wherein the semi-synthetic hydrocarbon feed has an aromatics content of below 0.5 mass %.

15. The process of claim 13, wherein the inert medium is nitrogen.

16. The process of claim 13, wherein the inert medium is steam.

17. The process of claim 13, wherein the lower olefins are selected from the group consisting of ethylene, propylene, butadiene, and mixtures thereof.

18. The process of claim 13, wherein the step of thermally cracking is conducted at a temperature of from 400° C. to 1200° C. and at a pressure of from 0.1 bar absolute pressure to 20 bar absolute pressure.

19. The process of claim 13, wherein the step of thermally cracking is conducted in a thermal cracking unit at a residence time of from 50 ms to 1000 ms.
